(19) United States
(12) Patent Application Publication
(10) Pub. No.: US 2003/0153866 A1
(43) Pub. Date: Aug. 14, 2003

(54) Self-propelled, intraluminal device with hollow, cylindrical head and method of use

(76) Inventors: Gary L. Long, Gerards Cross (GB); Kurt R. Bally, Lebanon, OH (US); David Stefanchik, Mason, OH (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/281,930

(22) Filed: Oct. 28, 2002

Related U.S. Application Data
(60) Provisional application No. 60/344,426, filed on Nov. 9, 2001.
(60) Provisional application No. 60/344,429, filed on Nov. 9, 2001.

(51) Int. Cl. A61M 1/00

(52) U.S. Cl. 604/28

(57) ABSTRACT

A medical device is provided for use in the lumen of a patient, such as in the Gastro-Intestinal Tract (GI Tract). The medical device can include a through channel for permitting passage of solid material, such as fecal material in the GI tract. In one embodiment, the device includes a self propelled capsule with a through channel, and a balloon disposed inside of the channel of the capsule. When inflated, the balloon provides occlusion of the channel.

This application claims priority to the following provisional patent applications: “Method for Providing Access to Luminal Tissue”, Serial No. 60/344,426, filed Nov. 9, 2001 in the name of Long et al.; and “Luminal Propulsive Device Having a Generally Continuous Passageway”, Serial No. 60/344,429, filed Nov. 9, 2001 in the name of Long et al.

### FIELD OF THE INVENTION

The present invention relates to a medical device that moves within a lumen of a patient's body.

### BACKGROUND

A physician typically accesses and visualizes tissue within a patient's gastrointestinal (GI) tract with a long, flexible endoscope. For the upper GI, a physician may insert a gastroscope into the sedated patient's mouth to examine and treat tissue in the esophagus, stomach, and proximal duodenum. For the lower GI, a physician may insert a colonoscope through the sedated patient's anus to examine the rectum and colon. Some endoscopes have a working channel, typically about 2.5-3.5 mm in diameter, extending from a port in the handpiece to the distal tip of the flexible shaft. A physician may insert medical instruments into the working channel to help diagnose or treat tissues within the patient. Physicians commonly take tissue biopsies from the mucosal lining of the GI tract using a flexible, biopsy forceps through the working channel of the endoscope.

Insertion of a flexible endoscope, especially into the colon, is usually a very time-consuming and uncomfortable procedure for the patient, even when sedated with drugs. A physician often needs several minutes to push a flexible endoscope through the convoluted sigmoid, descending, transverse, and ascending portions of the colon. The physician may diagnose and/or treat tissues within the colon either during insertion or removal of the endoscope. Often the flexible endoscope “loops” within the colon, such as at the sigmoid colon or at the splenic flexure of the colon, so that the inserted length of the endoscope is longer than the portion of colon containing it. Depending on the anatomy of the patient and the skill of the physician in manipulating the flexible endoscope, some portions of the colon may be unexamined, thus increasing the risk of undiagnosed disease.

Given® Engineering LTD, Yoqneam, Israel, sells a device in the U.S. called the M2A™ Swallowable Imaging Capsule. The device contains a tiny video camera, battery, and transmitter. It is propelled through the gastrointestinal tract by natural peristalsis. The device is currently used for diagnostic purposes and passes through the intestinal tract with a velocity determined by the natural, peristaltic action of the patient's body. World Publication WO 0108548A1 filed by C. Mosse, et al. describes a self-propelling device adapted to travel through a passage having walls containing contractile tissue. The applicants disclose that the device is particularly useful as an enteroscope and may also carry objects such as feeding tubes, guide wires, physiological sensors or conventional endoscopes within the gut. A summary of other alternatives to push endoscopy can be found in “Technical Advances and Experimental Devices for Enteroscopy” by C. Mosse, et al, published in Gastrointestinal Endoscopy Clinics of North America, Volume 9, Number 1, January 1999: pp. 145-161.

Often during colonoscopy, the physician finds that the patient has been inadequately prepared for the procedure, and a large amount of feces and other matter may be obstructing the passage of a flexible endoscope into the colon. A device and method for either proceeding with the examination or for cleaning the colon immediately prior to the examination would save overall time and costs for both the physician and the patient. What is needed, therefore, is a self-propelled, intraluminal device that includes means for using the device in a lumen containing obstructive matter such as feces, or for thoroughly cleaning such matter from the lumen prior to endoscopic examination.

### SUMMARY OF THE INVENTION

In one embodiment, the present invention provides an apparatus, such as a capsule, adapted for movement through a bodily lumen, such as the gastro-intestinal (GI) tract. The apparatus comprises a channel passing longitudinally through the apparatus. The through channel is sized and shaped to pass solid material, such as fecal material in the GI tract. The device can include an inflatable member for occluding the through channel so that the solid material can be withdrawn from the lumen when the apparatus is removed from the body. The apparatus can include one or more electrodes for providing electrical stimulation of lumen tissue for moving the apparatus through the lumen. The apparatus can further include a recess in its outer surface, and one or more seals for separating lumen tissue positioned at the recess from adjacent portions of the lumen.

The present invention can also provide a method for removing solid material, such as fecal material, from a lumen. By way of example, the method can be used as part of a bowel preparation procedure prior to a subsequent surgical or other medical procedure. In one embodiment, the method includes the steps of moving an apparatus sized and shaped to pass through the GI tract, such as by electrical stimulation of GI tissue, expanding a portion of the apparatus, such as by inflation, and withdrawing the apparatus from GI tract to remove the material from the GI tract.

### BRIEF DESCRIPTION OF THE DRAWINGS

We have set forth the novel features of the invention with particularity in the appended claims. To fully understand the invention, however, please refer to the following description and accompanying drawings.

FIG. 1 is a cross sectional view of a wall 14 of a hollow organ such as the colon.

FIG. 2 is a perspective view of a medical device 101 of the present invention, including an end view and a side view of a capsule 100, side views of an umbilicus 140, a valve 42, and an inflater 40, and a schematic view of a control unit 20.

FIG. 3 is a sectional view of capsule 100 shown in FIG. 2 positioned inside of a bodily lumen 15, wherein capsule 100 includes a channel 102 for passage of solid matter 16.

FIG. 4 is a sectional view of capsule 100 shown in FIG. 2 positioned inside of a bodily lumen 15, wherein a balloon 108 is shown in an inflated configuration and occluding channel 102 to prevent the passage of solid matter 16.

FIG. 5 is an end view of a capsule 200, which includes a plurality of distal electrodes 212 and a channel 202.

FIG. 6 is a side view of capsule 200 shown in FIG. 5, and also including a plurality of proximal electrodes 210.

FIG. 7 is a sectional view of capsule 200 shown in FIG. 6, showing an umbilicus 240 attached to the inside of channel 202, and including a balloon 208 shown in a deflated configuration.

FIG. 8 is an end view of a capsule 300, which includes a plurality of distal electrodes 312 and a channel 302, wherein distal electrodes 312 are flexibly mounted.

FIG. 9 is a side view of capsule 300 shown in FIG. 8, and also including a plurality of distal electrodes 310, wherein proximal electrodes 310 are flexibly mounted.

FIG. 10 is a sectional view of capsule 300 shown in FIG. 9, showing an umbilicus 340 attached to the inside of channel 302 and including a balloon 308 shown in a deflated configuration.

FIG. 11 is an end view of a capsule 400, which includes a leading end 404 having a plurality of struts 403 spanning a channel 402.

FIG. 12 is a side view of capsule 400 shown in FIG. 11, showing leading end 404 and a trailing end 406 separated by an expandable body 405, and an umbilicus 440 attached to trailing end 406.

FIG. 13 is a sectional view of capsule 400 of FIG. 12, showing a conduit 416 longitudinally positioned inside of channel 402 and connecting trailing end 406 to leading ends 404, and a balloon 408 shown in a deflated configuration and mounted on conduit 416.

FIG. 14 is a side view of capsule 400 of FIG. 13 positioned inside of bodily lumen 15 and in a contracted configuration, thus allowing passage of solid matter 16 as capsule 400 moves in a forward (left) direction.

FIG. 15 is a sectional view of capsule 400 of FIG. 14 positioned inside of bodily lumen 15 and in an expanded configuration, wherein balloon 408 is shown in an inflated configuration, thus occluding channel 408, so that the operator may pull an umbilicus 440 to remove solid matter 16 from bodily lumen 15.

FIG. 16 is schematic view of a medical device 501, another embodiment of the present invention, and includes the same elements as medical device 101 of FIG. 1, but also includes a fluid source 32, a vacuum source 30, a fluid valve 34, and a fluid line 36.

FIG. 17 is a perspective view of a capsule 500, which includes a recess 515 with a port 517, a plurality of distal electrodes 512, a plurality of proximal electrodes 510, and an umbilicus 540.

FIG. 18 is a perspective view of a capsule 600, which includes a recess 615 with a port 617, a plurality of distal electrodes 612 flexibly mounted, a plurality of proximal electrodes 610 flexibly mounted, and an umbilicus 640.

FIG. 19 is a sectional view of capsule 500 of FIG. 17, shown in a non-sealing configuration while recess 515 is positioned over a tissue structure 13 inside of a working space 516.

FIG. 20 is a sectional view of capsule 500 of FIG. 19, shown in a sealing configuration while working space 516 is filled with a fluidic agent 31.

FIG. 21 is a sectional view of capsule 500 of FIG. 20, shown as a medical device 580 may be used inside of working space 516.

FIG. 22 is an exploded, perspective view of capsule 500 of FIG. 17, an endoscope adapter 600, and an endoscope 680.

### DETAILED DESCRIPTION OF THE INVENTION

The present invention is a self-propelled intraluminal medical device including one or more of the improvements previously listed. By way of example, the present invention is illustrated and described for application in the colon of a human patient. However, the present invention is applicable for use in the bodily lumens of other hollow organs in humans and in other mammals.

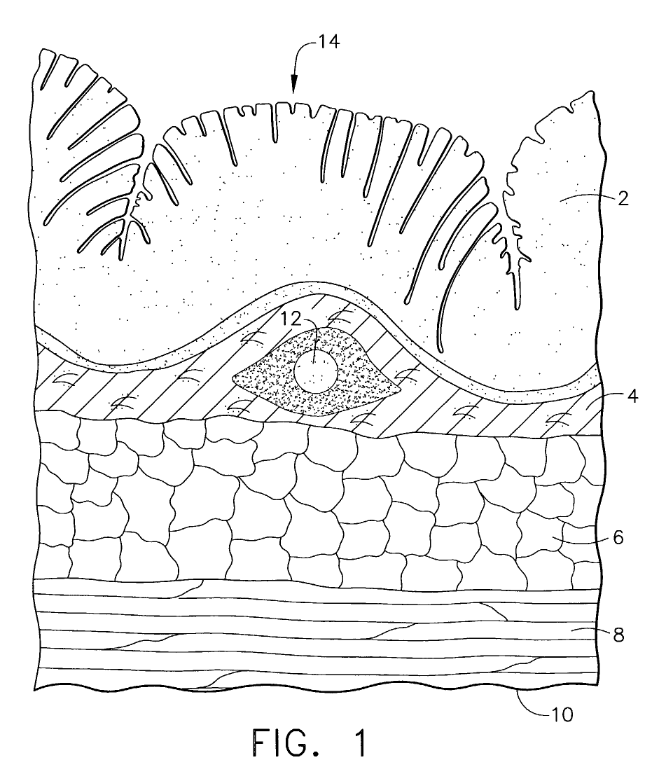

FIG. 1 shows a section of a wall 14 of the mammalian colon, and includes a mucosal layer 2, a submucosal layer 4 (shown with a lymph node 12), a circular muscular layer 6, a longitudinal muscular layer 8, and a serosa 10. Natural peristalsis is a progressive wavelike contraction of wall 14 that occurs involuntarily and is normally stimulated by distention of wall 14 from the contents within. Circular muscular layer 6 and longitudinal muscular layer 8 comprise the contractile tissue and contract when electrically stimulated, causing an instantaneous circumferential reduction of that portion of the lumen.

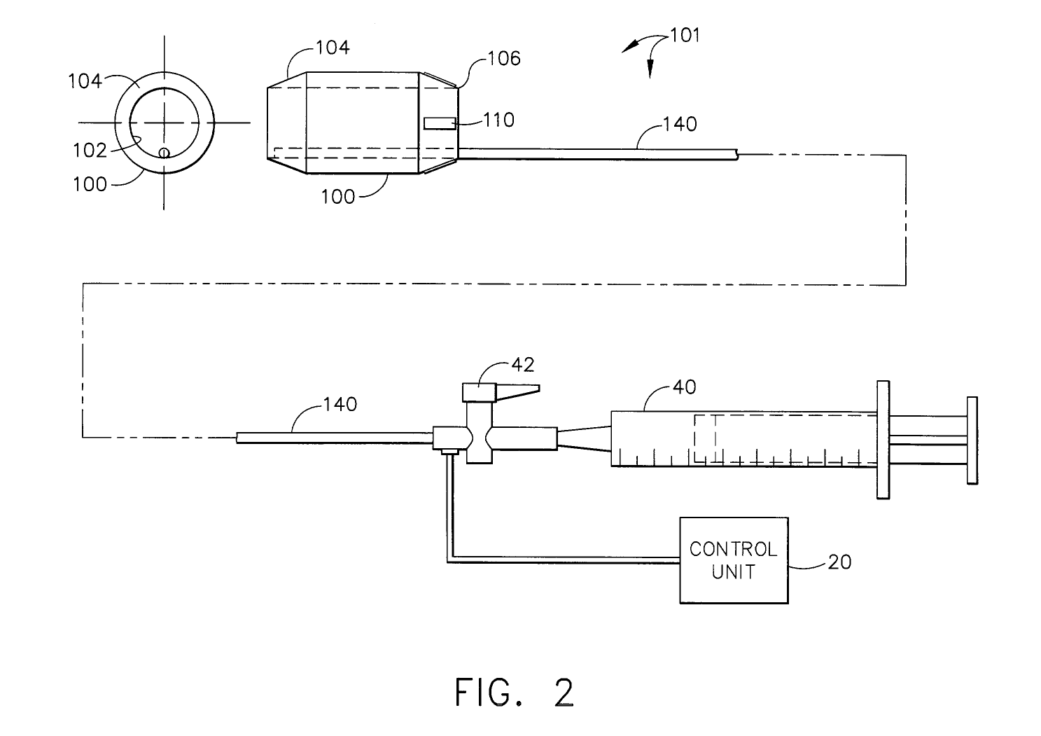

FIG. 2 illustrates a medical device 101 of the present invention and comprises a capsule 100, an umbilicus 140, a control unit 20, an inflater 40, and a valve 42. Capsule 100 has a leading end 104, a trailing end 106, and is sized to slide easily through the anus of the patient. In general, the outside of capsule 100 is smooth and streamlined for sliding easily through the colon. Trailing end 106 of capsule 100 is tapered so that when the colon constricts due to electrical stimulation, capsule 100 moves in a forward direction with attached umbilicus 140 trailing behind. Many other suitable shapes for capsule 100 are possible. Umbilicus 140 is flexible and is approximately as long as the flexible shaft of a colonoscope, which typically has a length of about 1.7 meters. Umbilicus 140 is preferably made from a thin wall flexible plastic or rubber tube suitable for transporting fluid between inflater 40 and capsule 100. Capsule 100 further includes a plurality of electrodes 110 that are mounted on trailing end 106 and electrically connected to control unit 20.

Control unit 20 provides electrical pulses to electrodes 110. At least one of electrodes 110 receives electrical pulses of a first electrical polarity, and the remaining electrodes receive electrical pulses of a second (opposite) electrical polarity. Control unit 20 comprises a frequency generator that provides at least one electrical waveform. Suitable waveforms include sinusoidal waves, square waves, triangular waves, and combinations. Control unit 20 also includes a constant current source, such as the Stimulus Isolator commercially available from World Precision Instruments of Sarasota, Fla. Control unit 20 allows the operator to activate and deactivate electrical stimulation to the colon, thus controlling the intraluminal propulsion of capsule 100. Control unit 20 also allows the operator to control the electrical pulse frequency of the stimulation, which may be generally uniform or varying. A suitable pulse frequency is approximately in the range of 5 to 20 Hz, but can be as high as approximately 1000 Hz. Control unit 20 also allows the operator to control electrical stimulation current amplitude. A suitable electrical stimulation current amplitude is approximately in the range of 10 to 50-mA, but can be as high as about 100-mA. However, it is also possible for waveform, frequency, and current amplitude to operate according to predetermined values set in control unit 20, therefore not requiring operator adjustment during the medical procedure. One particularly suitable electrical stimulation type is a half duty cycle, 15 Hz, 30-mA square wave.

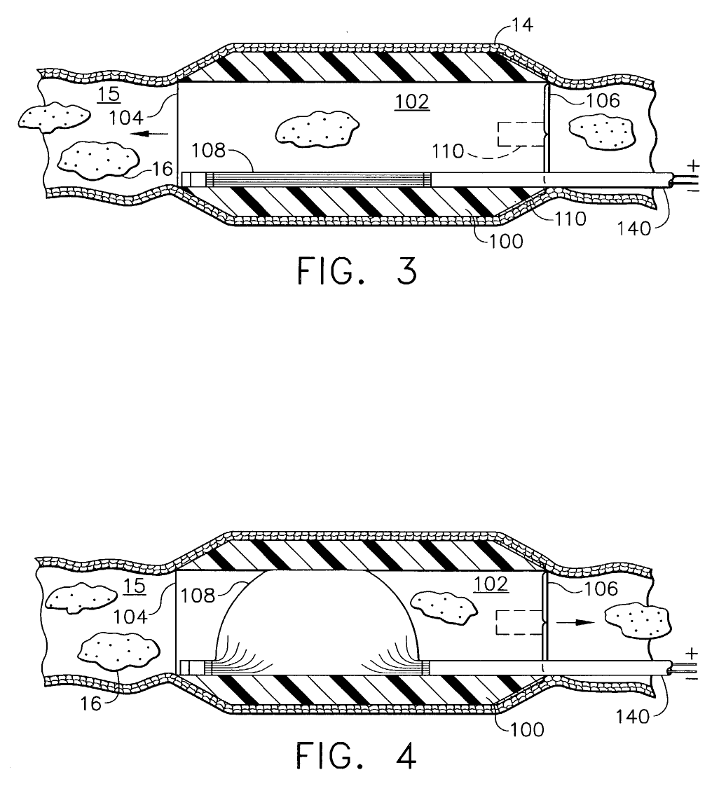

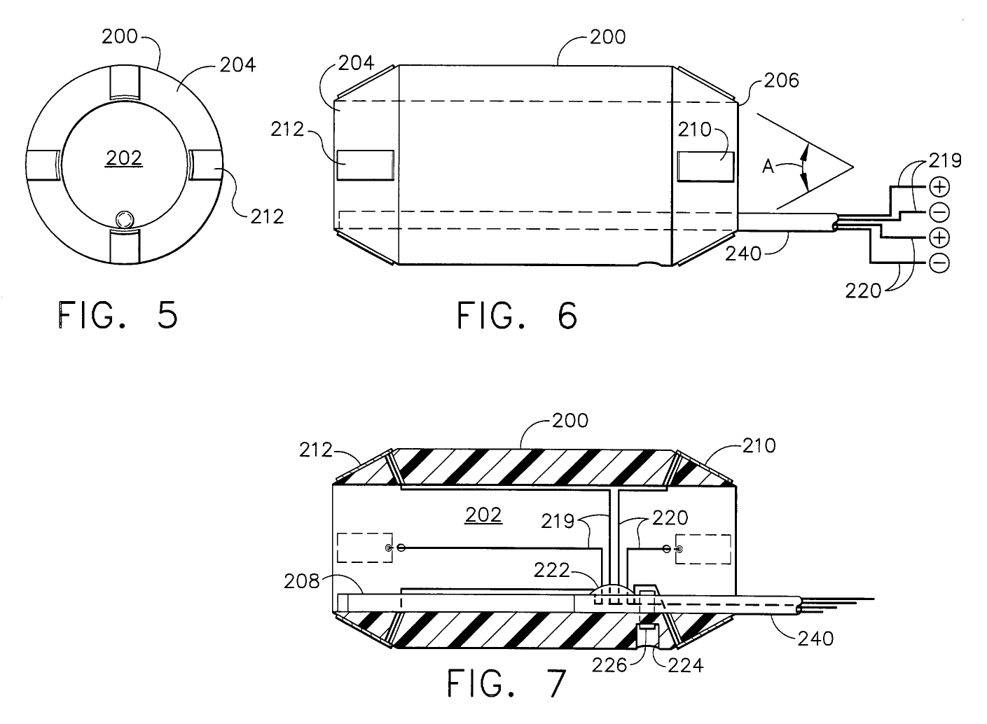

FIG. 3 and FIG. 4 are sectional views of capsule 100 while positioned inside of a bodily lumen 15, which is the lumen of the colon in this example. Capsule 100 can include a through channel 102. Through channel 102 can be sized and shaped to be able to pass solid material, such as fecal material, through the capsule 100. The channel 102 can have a generally cylindrical shape (generally circular when viewed on end, as in FIG. 5), though other shapes may be suitable. The channel 102 can have a maximum radial dimension (such as when viewed as in FIG. 5) which is at least about one-half the maximum radial dimension of the capsule 100 (e.g. channel 102 can have diameter which is at least about half the maximum diameter of the capsule 100); more particularly, the channel 102 can have a maximum radial dimension which is at least about two-thirds the maximum radial dimension of the capsule 100. By way of example, the channel 102 can have a diameter of at least about one half inch; more particularly the channel 102 can have a diameter of at least about three quarters of an inch.

In FIG. 3, solid matter 16 passes through a channel 102 of capsule 100 as capsule 100 and umbilicus 140 move in a forward (left) direction. A balloon 108 mounted on the distal end of umbilicus 140 inside of channel 102 is shown in a deflated configuration, thus allowing passage of solid matter 16 through capsule 100. Electrodes 110 contact wall 14 of the colon as electrical pulses from control unit 20 (FIG. 2) electrically stimulate the contractile tissue in wall 14. The portion of wall 14 that surrounds trailing end 106 of capsule 100 constricts, and capsule 100 “selfpropels” in the forward direction, which for this example, is against the natural, peristaltic direction. In FIG. 4, balloon 108 is shown in the inflated configuration, thus occluding channel 102 and preventing passage of solid matter 16 through capsule 100. While control unit 20 is deactivated, the operator may pull gently on umbilicus 140 to move capsule 100 in a reverse (right) direction, while at the same time removing solid matter 16 that is on the proximal side of capsule 100. As capsule 100 is pulled towards the anus, solid matter 100 and accompanying fluids may then be collected in an appropriate receptacle external to the patient. The bowel preparation is then completed, and the physician may next proceed with a conventional colonoscopy using a flexible endoscope to examine the interior walls of the colon.

FIG. 5 is an end view and FIG. 6 is side view of a capsule 200, which comprises a leading end 204 having a plurality of distal electrodes 212, and a trailing end 206 having a plurality of proximal electrodes 210. Capsule 200 may be used instead of capsule 100 as part of medical device 101 shown in FIG. 2. Distal electrodes 212 electrically connect to control unit 20 with a pair of wires 219. Proximal electrodes 210 electrically connect to control unit 20 with a pair of wires 220. When control unit 20 sends electrical pulses to proximal electrodes 210, capsule 200 moves in the forward direction. When control unit 20 sends electrical pulses to distal electrodes 212, capsule 200 moves in the reverse direction. When control unit 20 sends electrical pulses to both distal electrodes 212 and proximal electrodes 210, capsule 200 is held tightly in place by the constriction of colon around trailing end 206 and leading end 204.

FIG. 7 is a sectional view of capsule 200 of FIG. 6. The distal portion of umbilicus 240 attaches to capsule 200 with a clip 226 located in a recess 224. A balloon 208, shown in a deflated configuration, is mounted on the distal portion of umbilicus 240 and is fluidly connected to inflater 40 (FIG. 2). When balloon 208 is in the deflated configuration, solid matter 16 may pass through capsule 200. When balloon 208 is in the inflated configuration, channel 202 is occluded and solid matter 16 cannot pass through capsule 200. Wires 219 and 220 exit umbilicus 240 through a seal 222.

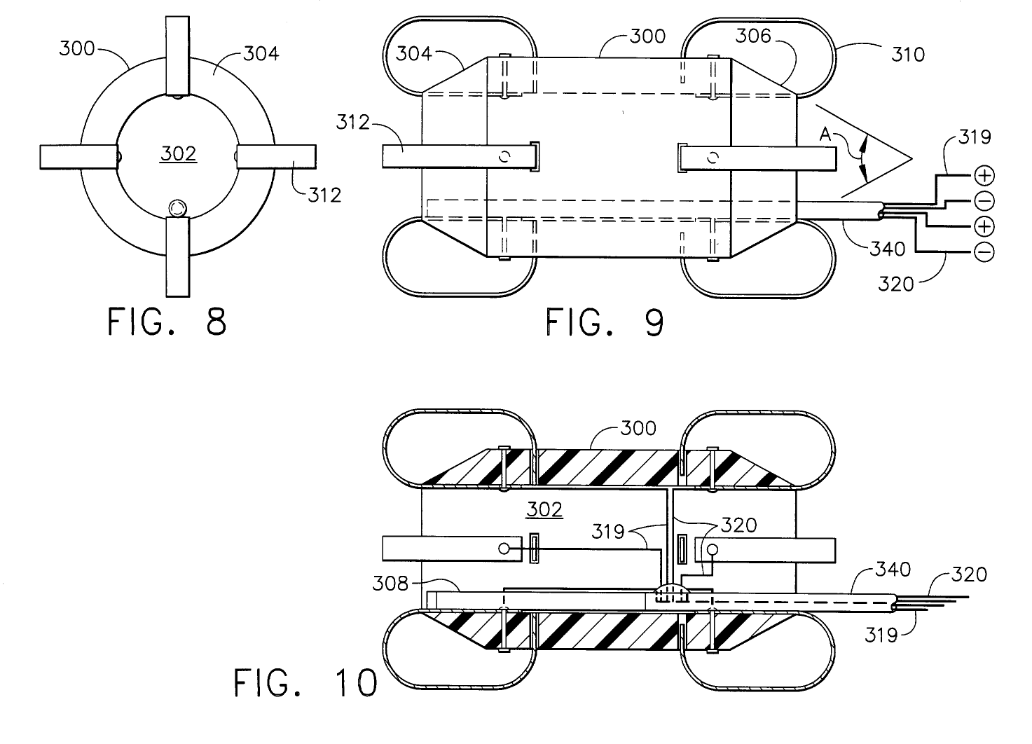

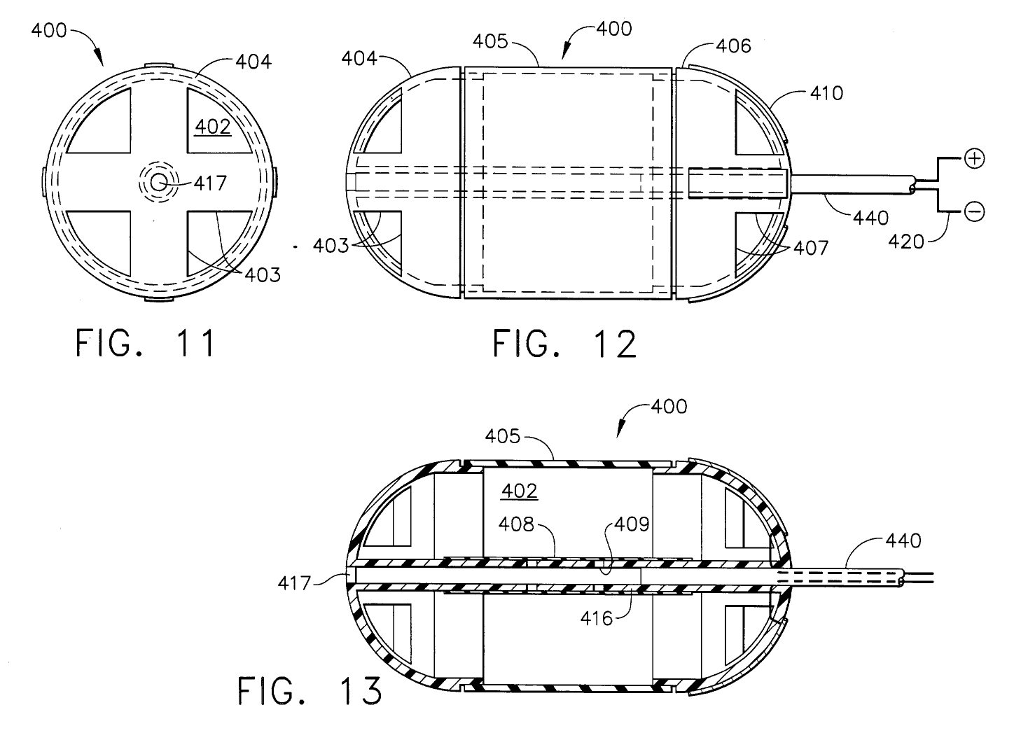

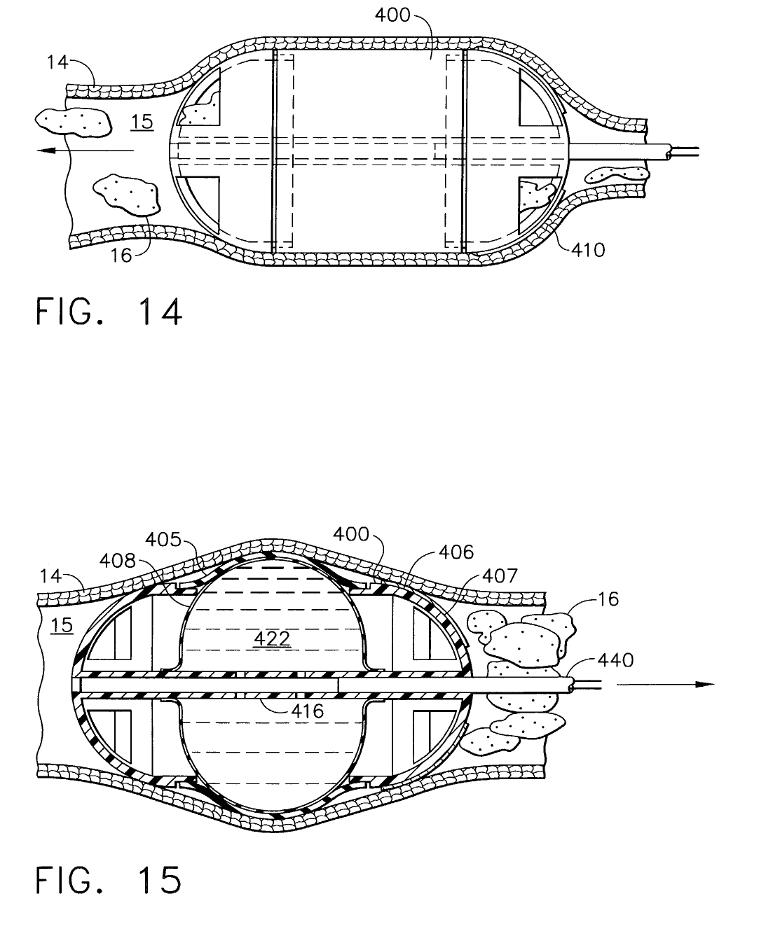

FIG. 8 is an end view and FIG. 9 is a side view of a capsule 300 that is generally the same as capsule 200 of FIG. 7, except that distal electrodes 312 are flexibly mounted on a leading end 304, and also except that proximal electrodes 310 are flexibly mounted on a trailing end 306. Each of distal electrodes 312 and proximal electrodes 310 are made from a spring metal flat that is formed into one of many possible shapes to allow deflection in the radial direction, thus providing intimate contact with bodily lumen walls of varying diameters to ensure optimal electrical stimulation. A pair of wires 319 electrically connect to distal electrodes 312, and a pair of wires 320 electrically connect to proximal electrodes 310. Control unit 20 provides electrical pulses in a similar manner as was describe for capsule 200 of FIG. 7. FIG. 10 is a sectional view of capsule 300. A balloon 308 is attached to the distal end of umbilicus 340 and is fluidly connected to inflater 40 (FIG. 2). The operator may occlude channel 302 and use capsule 300 for bowel preparation in a similar manner as was described for capsule 100 shown in FIG. 3 and FIG. 4.

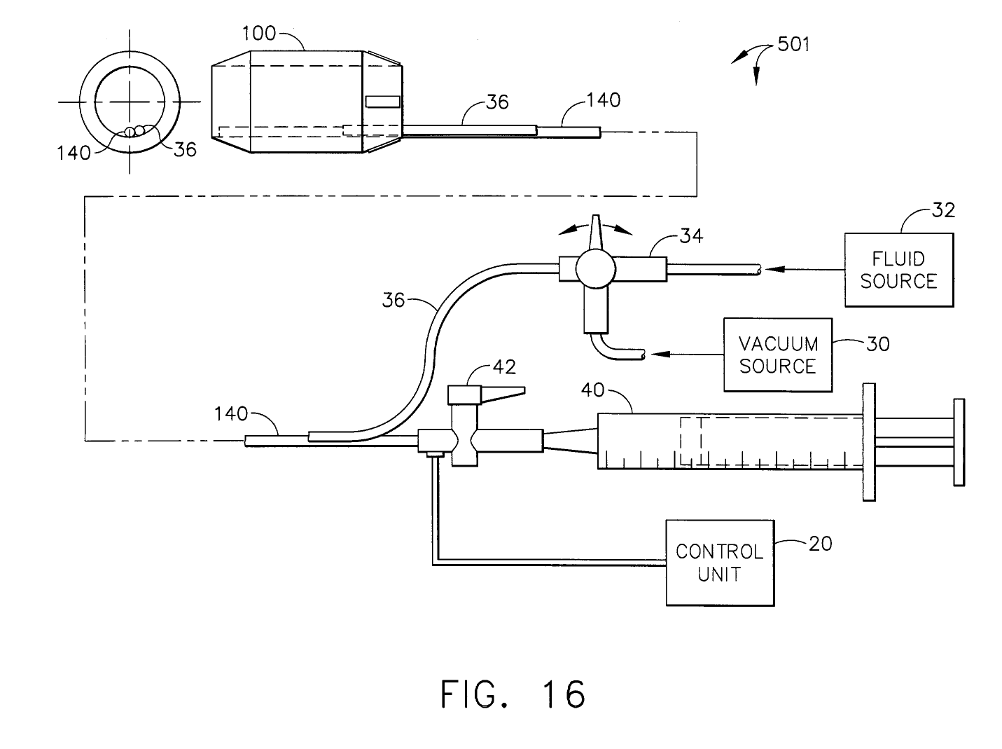

FIG. 11 is an end view and FIG. 12 is a side view of a capsule 400, which may be used in medical device 101 of FIG. 2 instead of capsule 100. Capsule 400 comprises a leading end 404, and expandable body portion 405, and a trailing end 406, which connects to an umbilicus 440. A plurality of electrodes 410 are mounted on trailing end 406 and are electrically connected to control unit 20 by wires 420. Leading end 404 includes a plurality of radial struts 403 that span the distal end of a channel 402. Trailing end 406 similarly includes a plurality of radial struts 407 that span the proximal end of channel 402. In the sectional view of capsule 400 in FIG. 13, a conduit 416 is disposed longitudinally in channel 402 and connects the centers of leading end 404 and trailing end 406. A balloon 408 is mounted around conduit 416 and is fluidly connected to inflater 40 (FIG. 2) via at least one port 409 in conduit 416. When the operator actuates inflater 40, balloon 408 inflates, occludes channel 402, and expands expandable body portion 405, and capsule 400 is in an expanded configuration. When the operator deflates balloon 408, channel 402 opens and capsule 400 resumes a contracted configuration. The distal end of umbilicus 440 connects to the proximal end of conduit 416. A pair of wires 420 run through conduit 416 and electrically connects electrodes 410 to control unit 20. Leading end 404 and the distal portion of conduit 416 are preferably injection molded as one piece from a rigid plastic such as polycarbonate. Trailing end 406 and the proximal portion of conduit 416 are also preferably injection molded as one piece from a rigid plastic. Expandable body portion 405 is preferably made from a length of rubber tube material and attaches to trailing end 406 and leading end 404 as shown. A plug 417 inserts into the distal end of conduit 416 to allow pressurization of balloon 408. Plug 417 may be removable to allow the operator to use conduit 416 and umbilicus 440 as a working channel for suction, irrigation, or the introduction of various types of medical instruments from outside the bodily lumen to the interior of the bodily lumen.

FIG. 14 and FIG. 15 depict how an operator may use medical device 101 (FIG. 2) with capsule 400 (FIG. 12) for bowel preparation. In FIG. 14 capsule 400 moves inside of bodily lumen 15 as the contractile tissue in wall 14 is electrically stimulated by electrodes 410. Solid matter 16 passes through capsule 400, which is in the contracted configuration. A sectional view of capsule 400 is shown inside bodily lumen 15 in FIG. 15 for when balloon 408 is inflated with a fluid such as saline and capsule 400 is in the expanded configuration. The outer diameter of expandable body portion 405 is increased substantially so that as the operator pulls on umbilicus 440, solid matter 416 on the proximal side of capsule 400 may be cleaned from the bodily lumen. Since umbilicus 440 attaches to the center of trailing end 406, and since radial struts 407 have a rounded profile, capsule 400 may be less likely to catch on protruding or uneven tissue structures (such as diverticula “pouches” in the colon) inside the bodily lumen than for the previous embodiments. During removal of capsule 400 from the bodily lumen, electrical stimulation may be deactivated, although electrical stimulation during removal would not normally be harmful to the patient, and may even be beneficial.

FIG. 16 illustrates another embodiment of the present invention, a medical device 501, which is very similar to medical device 101 of FIG. 2. Medical device 501 includes a capsule 100, an umbilicus 140, an inflater valve 42, an inflater 40, and a control unit 20. Medical device 501, however, includes the additional elements of a fluid source 32, a vacuum source 30, a fluid valve 34, and a fluid line 36. The operator may use medical device 501 to administer suction and irrigation from outside the bodily lumen to the inside of the bodily lumen. Fluid source 32 may comprise a fluid such as saline, water, a pharmaceutical agent, a surface anesthetic solution, or a cleaning agent, for example. Medical device 501 may be used with any of the capsule embodiments described herein.

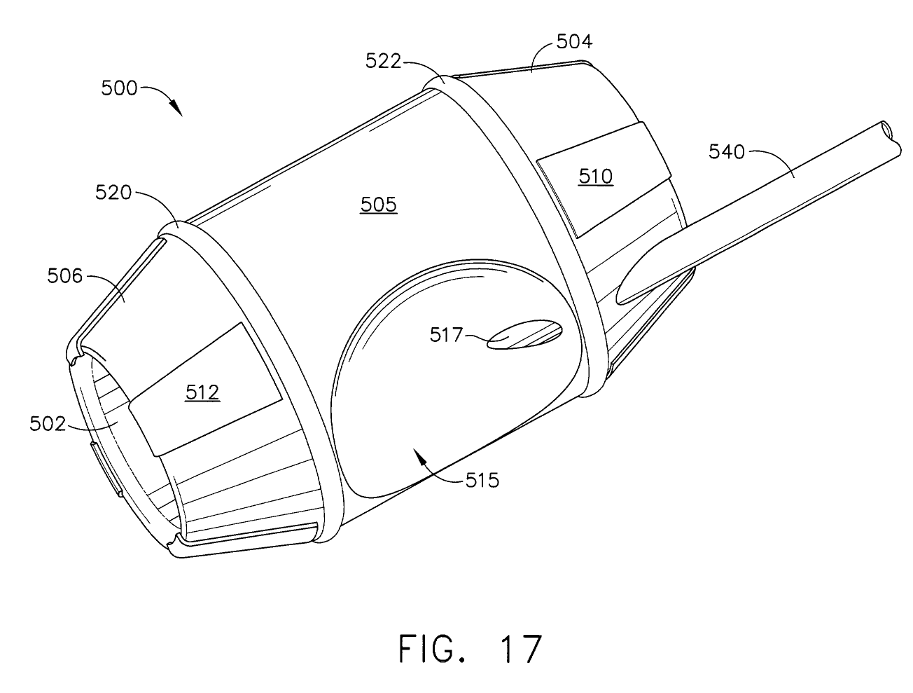

FIG. 17 is a perspective view of a capsule 500, which is very similar to capsule 200 of FIG. 6. Capsule 500 comprises a trailing end 506 with a plurality of proximal electrodes 510, a leading end 504 with a plurality of distal electrodes 512, a body portion 505, and a channel 502 for the passage of solid matter 16 as described for the previous embodiments. An umbilicus 540 attaches to trailing end 506. Capsule 500 also includes, however, a distal inflatable sealing ring 520, and a proximal inflatable sealing ring 522. Sealing rings 520 and 522 are disposed circumferentially around body portion 505. A recess 515 with a port 517 is positioned between sealing rings 520 and 522 on the outside of body portion 505. When capsule 500 is inside the bodily lumen, recess 515 creates a sealed working space over a portion of the wall of the bodily lumen. Port 517 provides the operator access to the inside of the working space with medical instruments, fluids, and the like, from outside the bodily lumen.

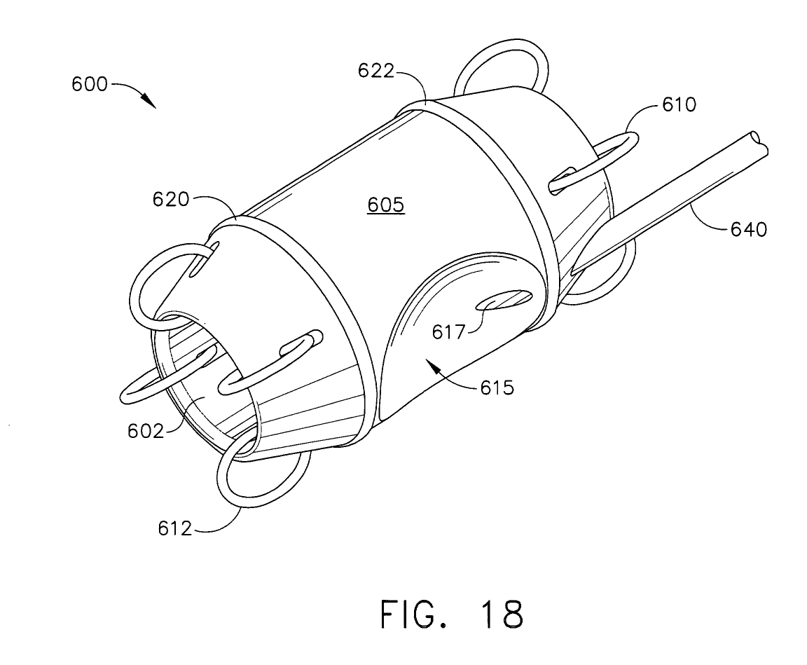

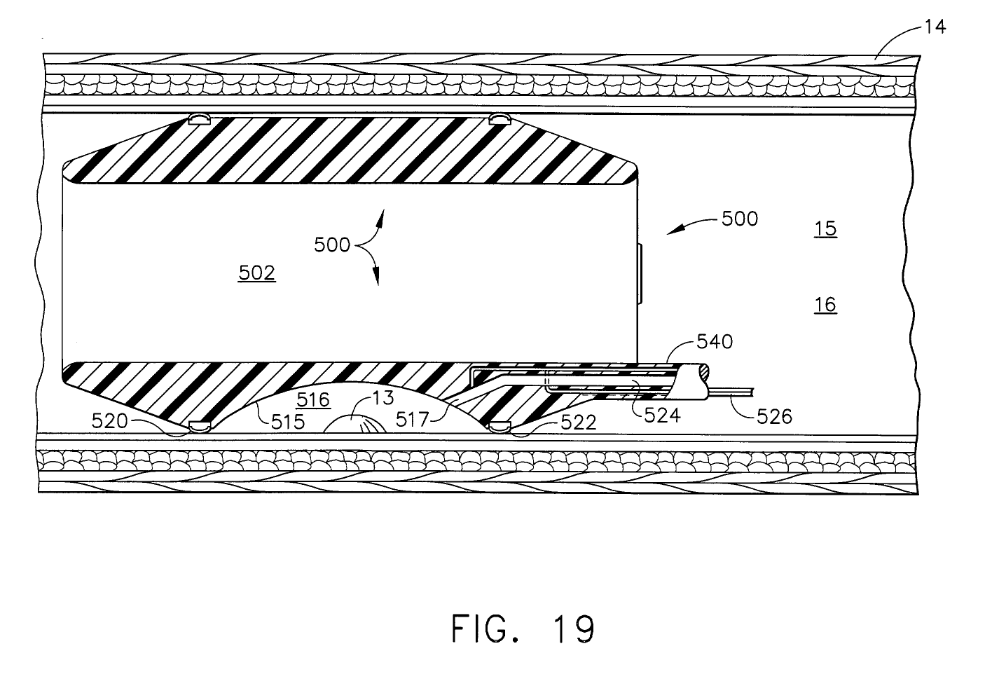

FIG. 18 is a perspective view of capsule 600, which is very similar to capsule 500 of FIG. 17. Capsule 600 comprises a trailing end 606, a leading end 604, a body portion 605, and a channel 602 for the passage of solid matter 16 as described for the previous embodiments. An umbilicus 640 attaches to trailing end 606. Capsule 600 also includes a distal inflatable sealing ring 620, a proximal inflatable sealing ring 622, a recess 615, and a port 617, as was described for capsule 500. Capsule 600 includes a plurality of proximal electrodes 610 and a plurality of distal electrodes 612 that comprise bare metal wire loops flexibly mounted on capsule 600 to improve electrical contact with the wall of the bodily lumen.

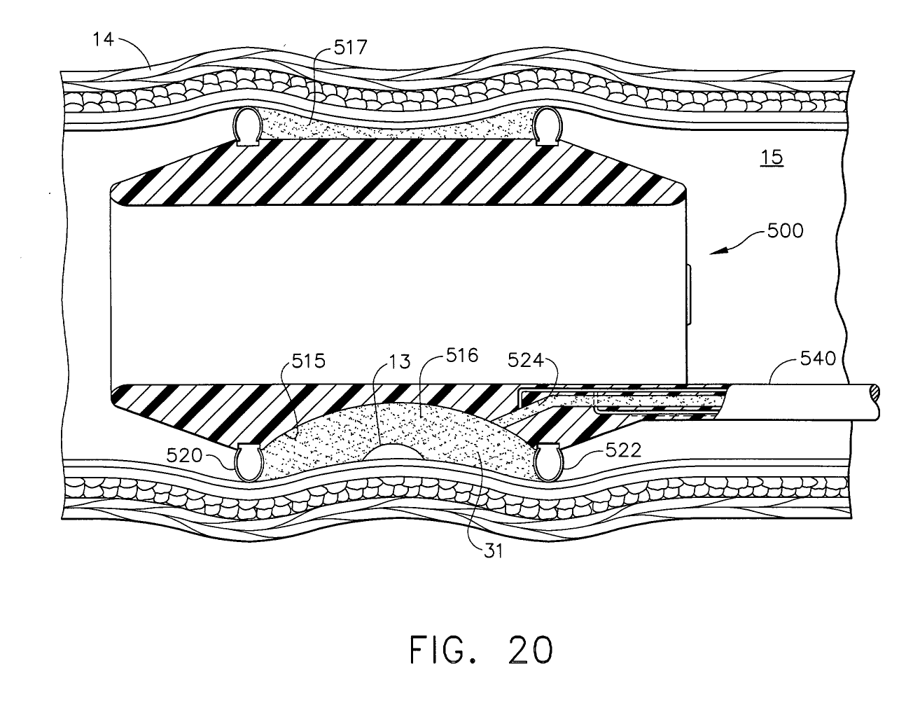

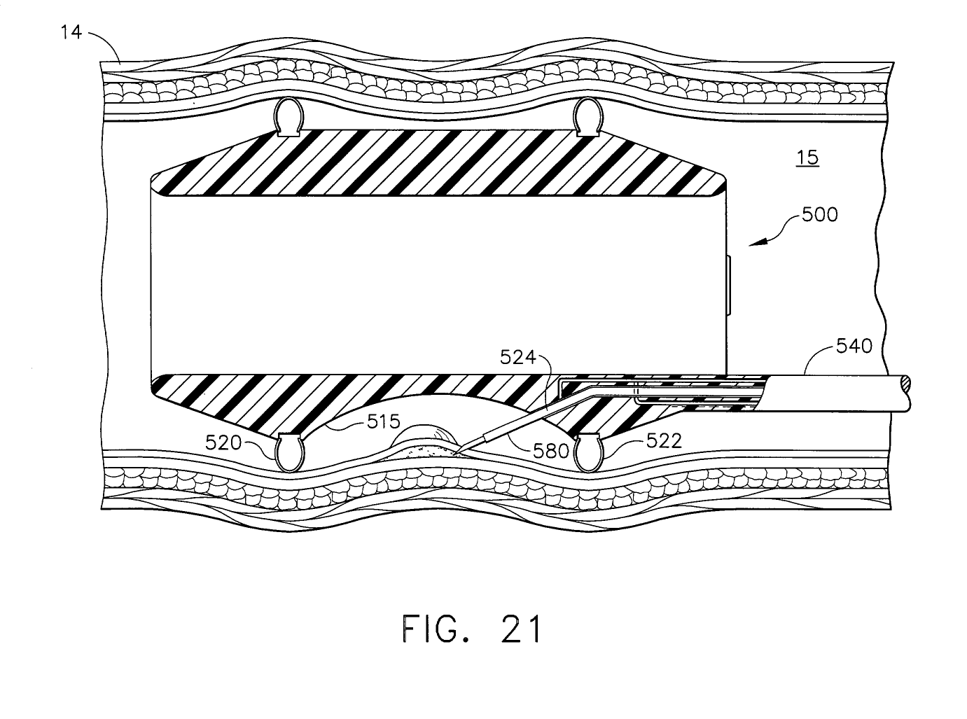

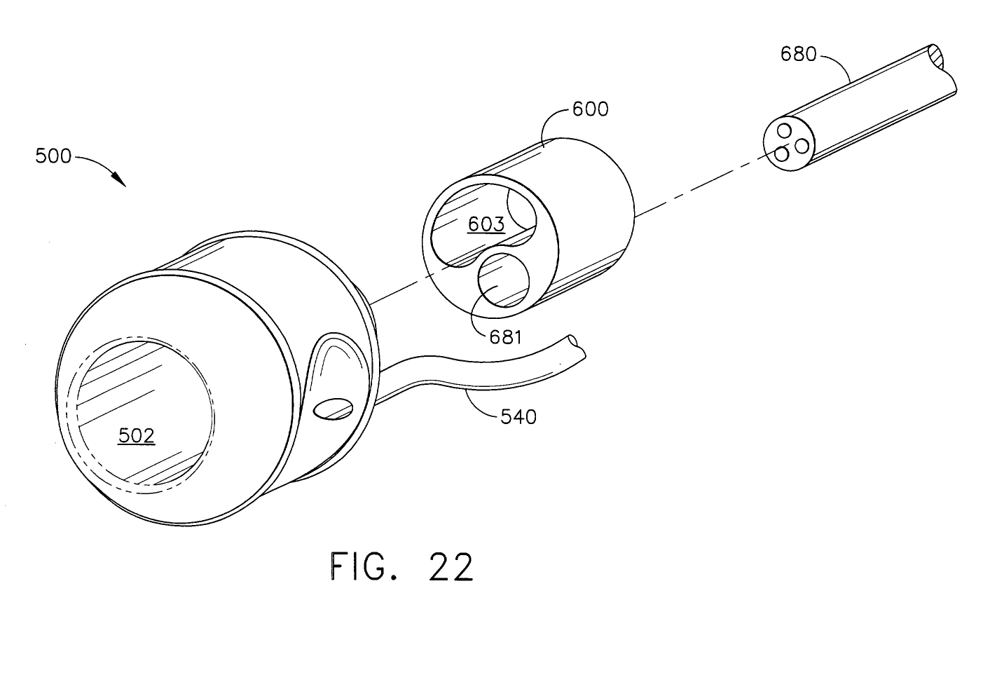

FIG. 19, FIG. 20, and FIG. 21 are sectional views of capsule 500 inside bodily lumen 15, and depict how an operator may use capsule 500 to treat a tissue structure 13 on wall 14. The operator advances capsule 500 within bodily lumen 15 using electrical stimulation as previously described until recess 515 is over the portion of wall 14 to be treated, creating a working space 516 over tissue structure 13, which may be a polyp, for example. The operator may rotate capsule 500 about the longitudinal axis by manually twisting umbilicus 540, to center tissue structure 13 inside working space 516. Visualization inside of working space 516 may be accomplished by numerous ways. For example, a very small diameter fiber optic visualization device (not shown) may be introduced through umbilicus 540 and port 517 to position recess 517 over tissue structure 13, then removed so that port 517 may be used for administering fluids, agents, and the like. It is possible also to install a small camera (CMOS, CCD) that is electrically connected to a display and signal processing unit (not shown) into capsule 500 to see directly into working space 516, or through a window in the wall of capsule 500. Another visualization means is to removably attach the distal end of a flexible endoscope to capsule 500 as shown in FIG. 22. A scope adapter 600 removably attaches to the inside of channel 502 of capsule 500. The distal end of endoscope 680 removably attaches to an adapter bore 681 of scope adapter 600. A passage 603 in adapter 600 allows the passage of solid matter as described for the previous embodiments so that the solid matter does not impede the advancement of capsule 500. Capsule 500 and adapter 600 may be constructed from a transparent material such as clear polycarbonate plastic. Also, endoscope 680 may be positioned within bore 681 so that working space 516 (FIG. 19) is within the field of view of endoscope 680. The operator may then view tissue structure 13 on a display during the procedure.

FIG. 20 illustrates sealing rings, 520 and 522, which are fluidly connected to inflater 40 (FIG. 16), in an inflated configuration, thus isolating a circumferential portion 517 of wall 14. A fluid 31 from fluid source 32 (FIG. 16) fills working space 516 and circumferential 517. Using capsule 500 in this way, the operator may apply a small amount of fluid 31 to only diseased tissue and adjacent tissue, rather than expose a much larger portion of wall 14 to fluid 31. FIG. 21 illustrates a medical instrument 524 inserted through port 517 for treatment of tissue structure 31. After treatment of tissue structure 13, the operator may deflate sealing rings, 520 and 522, and move capsule 500 within bodily lumen 15 as described for the other embodiments. A balloon may also be constructed into capsule 500 so that capsule 500 may also be used for bowel preparation as described for the previous embodiments.

While the present invention has been illustrated by description of several embodiments and while the illustrative embodiments have been described in considerable detail, it is not the intention of the applicant to restrict or in any way limit the scope of the appended claims to such detail. Further, various elements of the present invention can be equivalently described in terms of a means for accomplishing the elements' associated functions.

What is claimed is:

1. A medical device comprising:
an apparatus adapted for movement through a bodily lumen by stimulation of tissue, wherein the apparatus comprises a channel passing longitudinally therethrough, and wherein the channel is sized to pass solid material.

2. The device of claim 1 wherein the device is adapted to provide at least partial occluding of the channel.

3. The device of claim 2 comprising an inflatable member for providing at least partial occluding of the channel.

4. The device of claim 1 wherein the apparatus comprises at least one electrode for stimulating tissue.

5. The device of claim 1 wherein the device comprises an umbilicus associated with the apparatus.

6. The device of claim 1 wherein the channel has a maximum radial dimension which is at least half the maximum radial dimension of the apparatus.

7. The device of claim 1 wherein the channel has a maximum radial dimension which is at least two-thirds the maximum radial dimension of the apparatus.

8. The device of claim 1 wherein the channel has a diameter of at least about one-half inch.

9. The device of claim 1 wherein the channel has a diameter of at least about three-quarters of an inch.

10. The device of claim 1 further comprising a balloon disposed inside of said channel, said balloon fluidly connected to an inflater for inflating said balloon with a fluid in order to at least partially occlude said channel.

11. The device of claim 10 further comprising a valve for providing selective inflation and deflation of said balloon.

12. The medical device of claim 1 further comprising an expandable body portion disposed between a leading end and a trailing end of the apparatus.

13. The medical device of claim 1 comprising a first distal electrode having a first electrical polarity and a second distal electrode having a second electrical polarity, wherein said electrodes are operable for propelling the apparatus through the lumen.

14. The medical device of claim 13 wherein said first and second electrodes are flexibly mounted on a trailing end of said apparatus.

15. The medical device of claim 13 wherein said first and said second electrodes are flexibly mounted on a leading end of said apparatus.

16. The medical device of claim 1 wherein said apparatus comprises a capsule, said capsule comprising a body portion disposed between a trailing end and a leading end, and a first sealing ring and a second sealing ring mounted circumferentially on said body portion, wherein said first and second sealing rings are fluidly connected to an inflater and are inflatable to an inflated configuration to isolate the circumferential portion of the wall of the bodily lumen between said first and second sealing rings from the remainder of said bodily lumen.

17. The medical device of claim 16 further comprising a recess located on said body portion of said capsule between said first and second sealing rings, said recess having a port connected to a working channel in said capsule and said umbilicus, wherein said recess is positionable over a tissue structure on the wall of the bodily lumen and said recess creates a working space over the tissue structure.

18. The medical device of claim 1 further comprising an endoscope adapter for removably attaching the distal end of a flexible endoscope to said apparatus.

19. A medical device, the device comprising:
an apparatus sized and shaped to pass through the lower gastro-intestinal tract, the apparatus comprising a through channel extending the length of the apparatus; and
at least one electrode associated with the apparatus for stimulating tissue to propel the apparatus through the GI tract;
wherein the through channel is sized and shaped to permit passage of fecal material through the apparatus when the apparatus is propelled through the GI tract.

20. A method of removing material from a bodily lumen, the method comprising the steps of:
providing an apparatus sized and shaped to pass through the lumen, the apparatus having a through channel;
moving the apparatus through the lumen wherein material in the lumen upstream of the apparatus passes through the through channel in the apparatus to be positioned downstream of the apparatus;
at partially occluding the through channel to restrict further passage of material in the lumen through the through channel;
withdrawing the apparatus from the lumen.

21. The method of claim 21 wherein the step of moving the apparatus through the lumen comprises stimulating tissue in the lumen.

22. The method of claim 21 wherein the step of moving the apparatus through the lumen comprises electrically stimulating tissue in the lumen.

23. The method of claim 21 wherein the step of at least partially occluding the through channel comprises inflating a member associated with apparatus.

24. The method of claim 21 comprising moving the apparatus through the gastro-intestinal tract.

25. The method of claim 21 performed as a portion of a bowel preparation procedure prior to a subsequent medical procedure.

26 A method of removing fecal material from the lower gastro-intestinal tract, the method comprisng:
providing an apparatus sized and shaped to pass through the gastro-intestinal tract;
moving the apparatus through fecal material in the gastro-intestinal tract;
expanding a member associated with the apparatus; and
withdrawing the apparatus from the gastro-intestinal tract.

27. A medical device, the medical device comprising:
an umbilicus;
an apparatus associated with the distal end of the umbilicus, the apparatus sized and shaped to be positioned in a portion of the gastro-intestinal tract, the capsule having an outer surface facing the tissue of the gastro-intestinal tract;
wherein the apparatus comprises a recess disposed in the outer surface for providing working access to a portion of gastro-intestinal tract.

28. The device of claim 27 further comprising a tissue stimulator for moving the apparatus in the gastro-intestinal tract.

29. The device of claim 27 further comprising a working channel associated with umbilicus and communicating with the recess in the outer surface of the apparatus.

30. The device of claim 27 further comprising at least one seal disposed on the apparatus, the seal positioned to isolate tissue associated the recess in the outer surface of the apparatus.

31. The device of claim 30 comprising a first seal disposed upstream of the recess and a second seal disposed downstream of the recess.

32. A method of treating the wall of a bodily lumen, the method comprising the steps of:
providing an apparatus sized and shaped for movement through the lumen, wherein the apparatus has an outer surface and a recess disposed in the outer surface;
moving the apparatus in the lumen to position the recess in the outer surface over a portion of the lumen to be treated;
sealing the portion of the lumen to be treated from an adjacent portion of the lumen; and
treating the portion of the lumen.

33. The method of claim 32 wherein the step of moving the apparatus comprises stimulating lumen tissue to move the apparatus.

34. The method of claim 33 wherein the step of stimulating lumen tissue comprises electrically stimulating lumen tissue.

35. The method of claim 32 wherein the step of treating the portion of lumen comprises directing fluid into the recess in the outer surface of the apparatus.
